(19) United States
(12) Patent Application Publication
(10) Pub. No.: US 2024/0148804 A1
(43) Pub. Date: May 9, 2024

(54) INACTIVATED STRAINS OF BACTERIA, SUCH AS VIABLE BUT NON-CULTURABLE BACTERIA, COMPOSITIONS AND USE THEREOF

(71) Applicant: BLL INVEST S.R.L., Milano (IT)

(72) Inventor: Andrea BIFFI, Milano (IT)

(21) Appl. No.: 18/280,926

(22) PCT Filed: Apr. 1, 2022
(86) PCT No.: PCT/IB2022/053057

§ 371 Date: Sep. 7, 2023

(30) Foreign Application Priority Data
Apr. 1, 2021 (IT) 102021000008300

(51) Int. Cl. A61K 35/747; A61K 35/745; C12N 1/04; C12N 1/20; C12N 1/36; C12R 1/225

(52) U.S. Cl. CPC A61K 35/747; A61K 35/745; C12N 1/04; C12N 1/20; C12N 1/36; C12R 2001/225

(57) ABSTRACT

The present invention relates to inactivated microorganisms, preferably inactivated by gamma irradiation, wherein said inactivated microorganisms are viable but not culturable. Furthermore, the present invention relates to said inactivated microorganisms for use in a method of treating intestinal dysbiosis, inflammatory or functional gastrointestinal diseases, autoimmune diseases and diseases caused by pathogenic microorganisms.

The present invention relates to inactivated strains of bacteria, preferably inactivated by gamma irradiation, wherein said inactivated strains of bacteria are viable but non-culturable (abbreviated VBNC). In particular, the inactivated strains of bacteria of the present invention, preferably inactivated by gamma rays, substantially preserve the cell membrane characteristics of the non-inactivated strains of probiotic bacteria from which they are derived, including a substantially intact cell membrane.

Furthermore, the present invention relates to compositions comprising said inactivated strains of bacteria, preferably inactivated by gamma rays. Finally, the present invention relates to said inactivated strains of bacteria and compositions thereof for use in the treatment of intestinal dysbiosis, gastrointestinal disease, autoimmune disease, skin disease, respiratory tract disease, genito-urinary tract disease and others as defined in the context of the present description. In particular, said strains of bacteria and compositions thereof are for use in modulating cytokine profiles and/or inflammatory and/or immune pathways.

Currently available formulations of probiotic bacteria strains are typically stored at a temperature no higher than 45° C., and they are usually freeze-dried or stored in an aqueous medium at refrigeration temperatures (e.g. at about 4° C.), as at higher temperatures the viability of probiotic bacteria is reduced, as is the beneficial effect they can provide to the host organism when administered in suitable amount.

The technical problem that the present invention addresses and solves is providing inactivated strains of bacteria that can retain (at least in part) a functional characteristics (enzymatic and/or metabolic activity) and benefits for the host organism similar, or nearly similar, to the strains of probiotic bacteria from which they are derived and/or that can be stored, handled and processed over a wide temperature range (e.g. at temperatures from 10° C. to 35° C.-40° C.) without losing their functional characteristics.

The Applicant, after intensive and prolonged research and development activity, has identified, isolated and studied strains of bacteria inactivated by appropriate techniques, preferably inactivated by gamma irradiation, wherein said inactivated bacteria and compositions thereof are capable of providing a benefit to the host organism when administered in a therapeutically effective amount, in analogy to the corresponding strains of probiotic bacteria (viable and culturable) from which they are derived.

The inactivated bacterial strains of the present invention are viable but non-culturable bacterial strains, i.e., they are bacterial strains that substantially retain part of the functional characteristics [i.e., enzymatic and metabolic activities, and integrity of molecules present on the outer surface of the cell (Microbe-Associated Molecular Patterns, MAMPs, which mediate their immunomodulatory activity)] of the non-inactivated probiotic strains from which they are derived but are not capable of forming colony on solid medium. In particular, the inactivated bacterial strains of the present invention, preferably inactivated by gamma rays, substantially preserve the characteristics of the cell membrane of the viable non-inactivated strains from which they are derived, such as a cell membrane substantially capable of maintaining the trans-membrane electrical potential.

The inactivated strains of bacteria of the present invention, preferably inactivated by gamma rays, are capable of acting on surfaces and/or mucous membranes of various anatomical districts, such as, for example, skin, oral cavity, nasal epithelium, mucous membranes of vagina, respiratory tract or gastrointestinal tract.

In particular, the inactivated bacterial strains of the present invention, preferably inactivated by gamma rays, are able to adhere to the intestinal membrane, maintain the intestinal microbiota in a state of homeostatic equilibrium and/or strengthen the intestinal mucosal barrier, thereby limiting intestinal translocation by bacterial pathogens and, thus, the risk of secondary bacterial infections.

Furthermore, the inactivated bacterial strains of the present invention, preferably inactivated by gamma rays, and compositions thereof are capable of modulating cytokine and/or antiviral immune profiles and/or inflammatory/immune pathways in a manner favorable to host health, for example, by reducing expression levels of one or more pro-inflammatory markers and/or stimulating expression levels of one or more anti-inflammatory markers.

The above technical characteristics of the inactivated strains of bacteria of the present invention, preferably inactivated by gamma rays, make said strains of bacteria and compositions thereof capable of treating, in a preventive and/or curative manner, gastrointestinal disease or symptoms of an inflammatory and/or functional nature, autoimmune disease, musculoskeletal inflammatory disease, disease and/or symptom arising from a pathogenic microorganism, disease and/or symptom related to deregulation of serotonin re-uptake, skin disease, respiratory disease, genito-urinary disease and other diseases as may be described herein.

The bacteria strains inactivated, preferably by gamma rays, of the present invention and compositions thereof do not have any relevant side effects and/or have a high safety profile since they cannot reproduce and therefore cannot reach an excessive concentration or cause local or systemic infections (septicaemia).

The strains of bacteria inactivated, preferably by gamma rays, of the present invention and compositions thereof can be administered to all categories of subjects in need, including elderly, pregnant or lactating women, paediatric subjects (0-3 and/or 3-12 years), immunocompromised subjects, subjects in whom the administration of probiotic bacterial strains is not recommended (e.g. subjects suffering from pancreatitis, acute IBD, or other diseases wherein there is a risk of translocation and subsequent bacteremia), (healthy) athletes and (healthy) students.

Furthermore, the inactivated bacterial strains of the present invention, preferably inactivated by gamma rays, and compositions thereof are easy to prepare, can have a good and long shelf-life, can be stored at room temperature, e.g. from 15° C. to 30° C., are easy to handle and process, and are cost-effective.

These purposes and others, which will be evident from the following detailed description, are achieved by the bacterial strains, compositions and mixtures of the present invention by means of the technical features disclosed in the present description and claimed in the appended claims.

### BRIEF DESCRIPTION OF THE DRAWINGS

FIG. 1A represents an adhesion assay on a Caco-2 cell line of the combination of bacterial strains L. paracasei DG® (CNCM I-1572) and L. paracasei LPC-S01® (DSM 26760) non-inactivated (AminoAlta Product™), thermally inactivated and inactivated by gamma radiation.

FIG. 1B represents an adhesion assay on Caco-2 cell line of Bifidobacterium bifidum MIMBb23sg (DSM 32708) samples non-inactivated (live) and inactivated by gamma rays (at different kiloGray (kGy), units of absorbed dose of ionising radiation).

FIG. 2 is a comparison of the p-galactosidase activity of the combination of bacterial strains L. paracasei DG® (CNCM I-1572) and L. paracasei LPC-S01® (DSM 26760) not inactivated (AminoAlta Product™), thermally inactivated and inactivated by gamma radiation.

FIG. 3 is a comparison of the β-galactosidase activity of Bifidobacterium bifidum MIMBb23sg (DSM 32708) samples non-inactivated, thermally inactivated and inactivated by gamma rays (at different kiloGray (kGy), units of absorbed dose of ionising radiation).

FIG. 4 is a cytofluorimetric analysis of Bifidobacterium bifidum MIMBb23sg (DSM 32708) samples not inactivated, thermally inactivated and inactivated by gamma rays (at different kiloGray (kGy), units of absorbed dose of ionising radiation).

FIG. 5 is the assessment of β-galactosidase activity after 12 months.

FIG. 6 is the assessment of membrane integrity after 18 months.

### DETAILED DESCRIPTION OF THE INVENTION

An object of the present invention is at least one inactivated microorganism, such as a strain of bacteria or a yeast, wherein said inactivated bacteria or yeasts are viable but non-culturable, and wherein said at least one strain of bacteria or yeast belongs to the genus selected from Lactobacillus (including all genera identified in the article by Zheng et al. after the reclassification of the genus Lactobacillus, Zheng et al. Int. J. Syst. Evol. Microbiol., 70(4):2782-2858, 2020), Bifidobacterium, Bacillus (e.g., B. clausii, B. coagulans, B. subtilis, B. licheniformis, Bacillus cereus), Propionibacterium, Streptococcus (e.g., S. salivarius, S. intermedius, S. thermophilus,

S. diacetylactis, S. oralis, S. macedonicus), Lactococcus, Aerococcus, Enterococcus (e.g., E. mundtii, E. faecium), Escherichia (e.g., E. coli species), Pediococcus, Leuconostoc (e.g., L. mesenteroides), Propionibacterium, Saccharomyces (e.g., S. cerevisiae, including S. cerevisiae var. boulardii, Candida (e.g., C. pintolopesii species); preferably Lactobacillus and/or Bifidobacterium.

Based on a polyphasic approach, in the above-mentioned article Zheng et al. reported the reclassification of the genus Lactobacillus into 25 genera including the amended genus Lactobacillus, which includes host-adapted organisms that have been referred to as the group of Lactobacillus delbrueckii, Paralactobacillus and 23 new genera for which the names Holzapfelia, Amylolactobacillus, Bombilactobacillus, Companilactobacillus, Lapidilactobacillus, Agrilactobacillus, Schleiferilactobacillus, Loigolactobacilus, Lacticaseibacillus, Latilactobacillus, Dellaglioa, Liquorilactobacillus, Ligilactobacillus, Lactiplantibacillus, Furfurilactobacillus, Paucilactobacillus, Limosilactobacillus, Fructilactobacillus, Acetylactobacillus, Apilactobacillus, Levilactobacillus, Secundilactobacillus and Lentilactobacillus are proposed.

It should be noted that the bacterial strain L. paracasei DG® (CNCM I-1572) was redeposited on 2 Feb. 2022 as Lacticaseibacillus paracasei DG I1572 DSM 34154 after the reclassification of the genus Lactobacillus published by Zheng et al. in the scientific journal Int. J. Syst. Evol. Microbiol., 70(4):2782-2858, 2020. The two names mentioned above are interchangeable with each other because they always refer to the same strain of bacteria, An object of the present invention are also inactivated filamentous fungi, Archaea and protists (algae and protozoa).

According to one aspect, said inactivated (viable but non-culturable) microorganisms of the present invention may be strains of bacteria or yeasts belonging to the group of “next generation probiotics” (abbreviated NGP, as defined in the article by Lin et al., Medicine in Microecology, 3 (2020) 100017), such as, for example, microorganisms belonging to a species selected from the group comprising or, alternatively, consisting of: species belonging to the genus Bacteroides, Akkermansia muciniphila, Faecalibacterium prausnitzii, Christensenella minuta, species belonging to the genus Clostridium, Prevotella copri.

What is stated in this description with regard to strains of bacteria is also applicable to the mentioned yeasts.

The term “viable” means that strains of bacteria have enzymatic and/or metabolic activity and a substantially intact cell membrane (with maintenance of the trans-membrane electrical potential).

The term “non-culturable” means that the strains of bacteria are unable to form colonies on solid medium. In particular, the inactivated strains of bacteria of the present invention, preferably inactivated by gamma rays, have been subjected to DNA damage such that the bacterium is unable to reproduce.

An inactivated bacterial strain of the present invention, as a viable but non-culturable bacterial strain, is obtained by inactivation of a viable and culturable bacterial strain (probiotic bacterial strain) according to methods and equipment known to the skilled person in the art, such as, for example, by heat treatment (abbreviated “TT”, e.g. pasteurisation or sterilisation), high pressure (abbreviated HP), ultraviolet rays (abbreviated UV), irradiation with gamma rays (or gammation or ionising radiation), or sonication. Further examples of methods that could potentially be used for the inactivation of the bacterial strains of the present invention are: pulsed electric field (PEF), ohmic heating and supercritical CO₂, drying and pH changes.

Preferably, an inactivated strain of bacteria of the present invention, as a viable but non-culturable strain of bacteria, is obtained by inactivation of a viable and culturable strain of bacteria (probiotic strain of bacteria) by irradiation with gamma rays (or gammation or ionizing radiation), according to methodologies and equipment known to the skilled person in the art.

Furthermore, the viability and/or membrane integrity of said inactivated bacterial strain of the present invention is measured and assessed by methodologies and equipment known to the skilled person in the art, preferably by cytofluorimetry.

For example, using cytofluorimetry, it is possible to determine the cell composition of the sample in terms of live, damaged and dead cells.

The use of the cytofluorimeter can be considered a valid approach for measuring the different physiological states of bacterial cells after inactivation. In particular, the use of fluorescent dyes in cytofluorimetry allows the evaluation of both structural and functional biological parameters, for example: cell membrane integrity (fluorescent dyes: SYTO24™ and propidium iodide), intracellular metabolic activity (fluorescent dye: carboxyfluorescein diacetate), and/or membrane potential (fluorescent dye: DiOC6=3,3′-diesiloxyacarbocyanine iodide).

Gamma ray irradiation is a well-known technique consisting of exposing a product, in this case at least one strain of probiotic bacteria, to electromagnetic radiation, usually emitted by cobalt 60 (60Co) or, more rarely, cesium 137 (137Ce). In practice, it is possible to irradiate one or more strains of probiotic bacteria, preferably in lyophilised form, with gamma rays in doses sufficient to inactivate said one or more strains of probiotic bacteria; by way of example, a dosage of from 5 to 50 kiloGray (kGy) (e.g., 5 kGy, 10 kGy, 15 kGy, 20 kGy, 25 kGy, 30 kGy, 35 kGy, 40 kGy, or 45 kGy) may be used, preferably from 5 kGy to 15 kGy, until complete inactivation of at least one strain of bacteria according to the present invention. The skilled person in the art is able to determine the parameters and conditions necessary for said gamma ray inactivation (e.g., time and temperature and kGy) in order to obtain bacteria that are substantially viable (viability substantially similar to the viability of the strains before undergoing inactivation) but not culturable.

For example, the strains of bacteria of the present invention can be irradiated with gamma rays for a period of time from about 15 seconds to about 48 hours (e.g., about 1 minute, 15 minutes, 30 minutes, 60 minutes, 90 minutes, 2 hours, 4 hours, 8 hours, 12 hours, 16 hours, 24 hours, or 36 hours) and/or at a temperature from 5° C. to 50° C. (e.g., 10° C., 15° C., 20° C., 25° C., 30° C., 35° C., 40° C., or 45° C.), preferably from 20° C. to 35° C.

An inactivated strain of bacteria of the present invention, when administered in an appropriate amount, provides health benefits to the host in analogy to the non-inactivated (probiotic) strain of bacteria from which it is derived.

“Probiotics” are defined as live microorganisms that, when administered in adeguate amount, provide a health benefit to the host (FAO/WHO definition ‘live microorganisms which when administered in adequate amounts confer a health benefit on the host’).

The term “paraprobiotics” (or ghost probiotics) in the context of the present invention is in line with the definition given on page 271, right-hand column, of the Review entitled “The immunomodulatory properties of probiotic microorganisms beyond their viability (ghost probiotics: proposal of paraprobiotic concept” published in Genus Nutr. (2011), 6:261-274, by V. Taverniti and S. Guglielmetti). It specifically states: ‘To this end, we propose the use of the term “paraprobiotic” (or “ghost probiotics”), to be defined as “non-viable microbial cells (intact or broken) or crude cell extracts (i.e. with complex chemical composition), which, when administered (orally or topically) in adequate amounts, confer a benefit on the human or animal consumer”.

The term ‘paraprobiotics’ includes intact or non-intact non-viable (i.e. non-culturable) microbial cells or crude cell extracts that, when administered (orally or topically) in appropriate amounts, provide a health benefit to the host (similar to the viable, culturable microbial cells from which they are derived). Examples of paraprobiotics are strains of bacteria inactivated by heat (e.g. tindalised strains of bacteria), sonication (ultrasound), gammation (gamma rays), or lysates of strains of bacteria or extracts of strains of bacteria.

The term ‘postbiotics’ means any substance released or produced through the metabolic activity of the viable probiotic strain of bacteria, wherein said postbiotics, when administered (orally or topically) in an appropriate amount, provide a health benefit to the host (in analogy to the viable, culturable strain of bacteria from which they are derived). Examples of postbiotics are exopolysaccharides, parietal fractions, metabolites or metabolic bioproducts.

The inactivated bacterial strains of the present invention (preferably inactivated by gamma rays) of the genus Lactobacillus (and subsequent taxonomic reclassification of members of the genus itself) may belong to a species selected from: L. paracasei, L. acidophilus, L. rhamnosus, L. amylolyticus, L. amylovorus, L. alimentarius, L. aviaries, L. brevis, L. buchneri, L. casei, L. cellobiosus, L. coryniformis, L. crispatus, L. curvatus, L. delbrueckii, L. farciminis, L. fermentum, L. gallinarum, L. gasseri, L. helveticus, L. hilgardii, L. johnsonii, L. kefiranofaciens, L. kefiri, L. mucosae, L. panis, L. collinoides, L. paraplantarum, L. pentosus, L. plantarum, L. pontis, L. reuteri, L. sakei, L. salivarius and L. sanfranciscensis; preferably belonging to one species selcted from: Lacticaseibacillus paracasei (formerly Lactobacillus paracasei or Lactobacillus casei) and Lactiplantibacillus plantarum (formerly Lactobacillus plantarum); even more preferably Lacticaseibacillus paracasei.

The species nomenclature of the strains of bacteria belonging to the genus Lactobacillus has been amended after the reclassification of said genus, as reported in the article by Zheng et al., Int. J. Syst. Evol. Microbiol., 70(4):2782-2858 (2020). In the context of the present invention, the species nomenclature adopted after said reclassification is adopted.

The inactivated bacterial strains of the present invention (preferably inactivated by gamma rays) of the genus Bifidobacterium can belong to a species selected from: Bifidobacterium animalis, B. bifidum, Bifidobacterium breve, Bifidobacterium infantis, Bifidobacterium longum, Bifidobacterium adolescentis, Bifidobacterium catenulatum, Bifidobacterium angulatum, Bifidobacterium asteroides, Bifidobacterium boum, Bifidobacterium choerinum, Bifidobacterium coryneforme, Bifidobacterium cuniculi, Bifidobacterium denticolens, Bifidobacterium dentium, Bifidobacterium gallicum, Bifidobacterium gallinarum, Bifidobacterium indicum, Bifidobacterium inopinatum, Bifidobacterium lactis, Bifidobacterium magnum, Bifidobacterium merycicum, Bifidobacterium minimum, Bifidobacterium pseudocatenulatum, Bifidobacterium pseudolongum, Bifidobacterium pullorum, Bifidobacterium ruminantium, Bifidobacterium saeculare, Bifidobacterium subtile, Bifidobacterium thermacidophilum, Bifidobacterium thermophilum and Bifidobacterium tsurumiense; preferably belonging to a species selected from: Bifidobacterium breve, Bifidobacterium animalis subsp. lactis and Bifidobacterium bifidum; even more preferably Bifidobacterium bifidum.

All bacterial strains cited in the present invention have been deposited in accordance with the provisions of the Budapest Treaty. The Depositor of the bacterial strains described and/or claimed in the present patent application and the owner thereof hereby sin da subito express their consent to make all such strains available for the whole duration of the patent.

Said at least one inactivated strain of bacteria of the present invention (preferably inactivated by gamma rays) may be a strain of bacteria selected from the group comprising or, alternatively, consisting of:
(a) a strain of bacteria belonging to the species Lacticaseibacillus paracasei (formerly Lactobacillus paracasei, abbreviated L. paracasei) identified as L. paracasei DG® or L. casei DG® (registered trademark by SOFAR S.p.A.) and deposited at the National Collection of Cultures of Microorganisms of the Institut Pasteur in Paris with the accession number CNCM I-1572 (deposited on 5 May 1995 by SOFAR S.p.A as Lactobacillus casei ssp. casei with accession number CNCM I-1572 and subsequently reclassified as Lactobacillus paracasei CNCM I-1572 and then Lacticaseibacillus paracasei CNCM I-1572; it is specified that it is always and only the same strain of bacteria regardless of the designation Lactobacillus casei DG® CNCM I-1572 or Lactobacillus paracasei DG® CNCM I-1572 or Lacticaseibacillus paracasei DG® CNCM I-1572),
(b) a strain of bacteria belonging to the species Lacticaseibacillus paracasei (formerly Lactobacillus paracasei, abbreviated L. paracasei) identified as LPC-S01® and deposited at the Deutsche Sammlung von Mikroorganismen und Zellkulturen GmbH (DSMZ) as Lactobacillus paracasei with the accession number DSM 26760 (deposited on 11 Jan. 2013 by Sofar S.p.A, and applied for conversion of the deposit to a deposit in accordance with the Budapest Treaty on 15 May 2017; subsequently reclassified as Lacticaseibacillus paracasei DSM 26760; it is specified that it is always and only the same strain of bacteria regardless of the designation Lactobacillus paracasei LPC-S01® DSM 26760 or Lacticaseibacillus paracasei LPC-S01® DSM 26760),
(c) a strain of bacteria belonging to the species Bifidobacterium breve identified as Bifidobacterium breve BbIBS01 and deposited at the Deutsche Sammlung von Mikroorganismen und Zellkulturen GmbH (DSMZ) with deposit number DSM 33231 (deposited on 31 Jul. 2019 by Sofar S.p.A),
(d) a strain of bacteria belonging to the species Bifidobacterium breve identified as Bifidobacterium breve BbIBS02 and deposited at the Deutsche Sammlung von Mikroorganismen und Zellkulturen GmbH (DSMZ) with deposit number DSM 33232 (deposited on 31 Jul. 2019 by Sofar S.p.A),
(e) a strain of bacteria belonging to the species Bifidobacterium animalis identified as Bifidobacterium animalis subsp. lactis BlIBS01 and deposited at the Deutsche Sammlung von Mikroorganismen und Zellkulturen GmbH (DSMZ) with deposit number DSM 33233 (deposited on 31 Jul. 2019 by Sofar S.p.A),
(f) a strain of bacteria belonging to the species Lactiplantibacillus plantarum (formerly Lactobacillus plantarum, abbreviated L. plantarum) identified as LpIBS01 and deposited at the Deutsche Sammlung von Mikroorganismen und Zellkulturen GmbH (DSMZ) as Lactobacillus plantarum with deposit number DSM 33234 (deposited on 31 Jul. 2019 by Sofar S.p.A and subsequently reclassified as Lactiplantibacillus plantarum DSM 33234; it is specified that it is always and only the same strain of bacteria regardless of the designation Lactobacillus plantarum LpIBS01 DSM 33234 or Lactiplantibacillus plantarum LpIBS01 DSM 33234),
(g) a strain of bacteria belonging to the species Bifidobacterium bifidum identified as Bifidobacterium bifidum MIMBb23sg or BbfIBS01, or a derivative thereof, wherein said strain of bacteria has been deposited at the Deutsche Sammlung von Mikroorganismen und Zellkulturen GmbH (DSMZ) with deposit number DSM 32708 on 4 Dec. 2017 by Sofar S.p.A (MIMBb23sg and BbfIBS01 are synonymous and interchangeable identification names, and always indicate the same strain of bacteria),
(h) a strain of bacteria belonging to the species Lacticaseibacillus paracasei (formerly Lactobacillus paracasei, abbreviated L. paracasei) identified as m.biome LIVESKIN88 and deposited at the Deutsche Sammlung von Mikroorganismen und Zellkulturen GmbH (DSMZ) as Lactobacillus paracasei with accession number DSM 33788 (deposited on 20 Jan. 2021 by LAC2BIOME S.r.I., Italy; subsequently reclassified as Lacticaseibacillus paracasei DSM 33788; it is specified that it is always and only the same strain of bacteria regardless of the designation Lactobacillus paracasei m.biome LIVESKIN88 DSM 33788 or Lacticaseibacillus paracasei m.biome LIVESKIN88 DSM 33788).

It should be noted that the bacterial strain L. paracasei DG® (CNCM I-1572) was redeposited on 2 Feb. 2022 as Lacticaseibacillus paracasei DG I1572 DSM 34154 after the reclassification of the genus Lactobacillus published by Zheng et al. in the scientific journal Int. J. Syst. Evol. Microbiol., 70(4):2782-2858, 2020. The two designations mentioned above are interchangeable with each other because they always refer to the same strain of bacteria,

According to an embodiment, said at least one inactivated strain of bacteria of the present invention (preferably inactivated by gamma rays) is the strain L. paracasei DG® CNCM I-1572.

According to a further embodiment, said at least one inactivated strain of bacteria of the present invention (preferably inactivated by gamma rays) is a mixture of the strain L. paracasei DG® CNCM I-1572 and at least one strain of bacteria selected from the group comprising or alternatively consisting of: L. paracasei LPC-S01® DSM 26760, Bifidobacterium breve BbIBS01 DSM 33231, Bifidobacterium breve BbIBS02 DSM 33232, Bifidobacterium animalis subsp. lactis BlIBS01 DSM 33233, Lactobacillus plantarum LpIBS01 DSM 33234, Bifidobacterium bifidum MIMBb23sg (or BbfIBS01) DSM 32708, and L. paracasei m.biome LIVESKIN88 DSM 33788; preferably L. paracasei LPC-S01® DSM 26760 and/or Bifidobacterium bifidum MIMBb23sg (or BbfIBS01) DSM 32708.

For example, said at least one inactivated bacterial strain of the present invention (preferably by gamma ray) is a mixture of Lactobacillus paracasei strain DG® CNCM I-1572 and Lactobacillus paracasei LPC-S01 DSM 26760, preferably in a CFU or weight ratio (DG®: LPC-S01) of about 1:1.

For example, said at least one inactivated bacterial strain of the present invention (preferably by gamma ray) is a mixture of the L. paracasei strain DG® CNCM I-1572 and L. paracasei m.biome LIVESKIN88 DSM 33788, preferably in a CFU or weight ratio (DG®: m.biome LIVESKIN88) of about 1:1.

For example, said at least one inactivated strain of bacteria of the present invention is a mixture of the following strains of bacteria (preferably inactivated by gamma rays): (a) L. paracasei DG® CNCM I-1572 and/or (b) L. paracasei LPC-S01® DSM 26760, and further (c) Bifidobacterium breve BbIBS01 DSM 33231, (d) Bifidobacterium breve BbIBS02 DSM 33232, (e) Bifidobacterium animalis subsp. lactis BlIBS01 DSM 33233, (f) L. plantarum LpIBS01 DSM 33234 and optionally (g) Bifidobacterium bifidum MIMBb23sg=BbfIBS01 DSM 32708 and/or (h) L. paracasei m.biome LIVESKIN88 DSM 33788; preferably in a a CFU or weight ratio (DG®: BbIBS01: BbIBS02: BlIBS01: LpIBS01: MIMBb23sg) of about 1:1:1:1:1:1, or (DG®: LPC-S01: BbIBS01: BbIBS02: BlIBS01: LpIBS01: MIMBb23sg) of about 1:1:1:1:1:1:1.

According to a further embodiment, said at least one inactivated strain of bacteria of the present invention (preferably inactivated by gamma rays) is the strain Bifidobacterium bifidum MIMBb23sg (or BbfIBS01) DSM 32708, or alternatively a mixture of strain of Bifidobacterium bifidum MIMBb23sg (or BbfIBS01) DSM 32708 and strain of L. paracasei DG® CNCM I-1572 and/or strain of L. paracasei LPC-S01® DSM 26760 and/or strain of L. paracasei m.biome LIVESKIN88 DSM 33788, as inactivated strains (preferably by gamma ray).

It is an object of the present invention a composition (in short, composition of the invention) comprising (i) an M-mixture (in short, M-mixture of the invention) and, optionally, (ii) at least one pharmaceutically acceptable additive and/or excipient; wherein said mixture M comprises or, alternatively, consists of at least one inactivated strain of bacteria of the present invention (preferably inactivated by gamma rays), wherein said at least one strain of bacteria belongs to one of the genera listed above in the present description (preferably ex-Lactobacillus or Bifidobacterium); preferably wherein said at least one inactivated strain of bacteria of the present invention belongs to one of the species listed above in the present description (preferably selected from: Lactobacillus paracasei, Lactobacillus plantarum, Bifidobacterium breve, Bifidobacterium animalis subsp. lactis, Bifidobacterium bifidum); more preferably wherein said at least one inactivated strain of bacteria of the present invention is selected from the group comprising or, alternatively, consisting of: L. paracasei DG® CNCM I-1572, L. paracasei LPC-S01® DSM 26760, Bifidobacterium breve BbIBS01 DSM 33231, Bifidobacterium breve BbIBS02 DSM 33232, Bifidobacterium animalis subsp. lactis BlIBS01 DSM 33233, L. plantarum LpIBS01 DSM 33234, Bifidobacterium bifidum MIMBb23sg (or BbfIBS01) DSM 32708, L. paracasei m.biome LIVESKIN88 DSM 33788, and a mixture thereof.

Preferably, the mixture M of the composition of the invention comprises or, alternatively, consists of a strain of L. paracasei DG® CNCM I-1572 inactivated (preferably by gamma rays).

According to one aspect, said mixture M of the composition of the invention comprises or, alternatively, consists of an inactivated (preferably by gamma rays) strain of L. paracasei DG® CNCM I-1572 or, alternatively, a mixture of the inactivated (preferably by gamma rays) strain L. paracasei DG® CNCM I-1572 and at least one inactivated (preferably by gamma rays) strain of bacteria selected from the group comprising or, alternatively, consisting of: L. paracasei LPC-S01® DSM 26760, Bifidobacterium breve BbIBS01 DSM 33231, Bifidobacterium breve BbIBS02 DSM 33232, Bifidobacterium animalis subsp. lactis BlIBS01 DSM 33233, Lactobacillus plantarum LpIBS01 DSM 33234, Bifidobacterium bifidum MIMBb23sg (or BbfIBS01) DSM 32708, and L. paracasei m.biome LIVESKIN88 DSM 33788; preferably L. paracasei LPC-S01® DSM 26760 and/or Bifidobacterium bifidum MIMBb23sg or BbfIBS01 DSM 32708.

For example, the mixture M of the composition of the invention comprises, or alternatively consists of, inactivated (preferably by gamma ray) strains of L. paracasei DG® CNCM I-1572 and L. paracasei LPC-S01® DSM 26760, preferably in a CFU or weight ratio (DG®: LPC-S01®) of about 1:1.

For example, the mixture M of the composition of the invention comprises, or alternatively consists of, inactivated (preferably by gamma rays) strains of L. paracasei DG® CNCM I-1572 and L. paracasei m.biome LIVESKIN88 DSM 33788, preferably in a CFU or weight ratio (DG®: m.biome LIVESKIN88) of about 1:1.

Advantageously, the mixture M of the composition of the invention may comprise or alternatively consist of a strain of L. paracasei DG® CNCM I-1572 and a strain of L. paracasei LPC-S01® DSM 26760 or m.biome LIVESKIN88 DSM 33788 inactivated (preferably by gamma rays) and at least one strain of bacteria inactivated (preferably by gamma rays) selected from the group comprising or alternatively consisting of: Bifidobacterium breve BbIBS01 DSM 33231, Bifidobacterium breve BbIBS02 DSM 33232, Bifidobacterium animalis subsp. lactis BlIBS01 DSM 33233, L. plantarum LpIBS01 DSM 33234, and Bifidobacterium bifidum MIMBb23sg or BbfIBS01 DSM 32708; preferably Bifidobacterium bifidum MIMBb23sg (or BbfIBS01) DSM 32708.

Further examples of the composition of the invention, varying the mixture M, are given below.

Said mixture M of the invention may comprise or alternatively consist of the following inactivated (preferably by gamma ray) strains of bacteria: (a) L. paracasei DG® CNCM I-1572 and further a mixture of the strains of bacteria comprising (c) Bifidobacterium breve BbIBS01 DSM 33231, (d) Bifidobacterium breve BbIBS02 DSM 33232, (e) Bifidobacterium animalis subsp. lactis BlIBS01 DSM 33233, (f) L. plantarum LpIBS01 DSM 33234 and optionally (g) Bifidobacterium bifidum MIMBb23sg (or BbfIBS01) DSM 32708.

Said mixture M of the invention may comprise or alternatively consist of the following inactivated (preferably by gamma ray) strains of bacteria: (a) L. paracasei DG® CNCM I-1572 and/or (b) L. paracasei LPC-S01® DSM 26760 (or (h) DSM 33788 instead of (b) DMS 26760), and additionally a mixture of the bacterial strains comprising (c) Bifidobacterium breve BbIBS01 DSM 33231, (d) Bifidobacterium breve BbIBS02 DSM 33232, (e) Bifidobacterium animalis subsp. lactis BlIBS01 DSM 33233, (f) L. plantarum LpIBS01 DSM 33234 and optionally (g) Bifidobacterium bifidum MIMBb23sg=BbfIBS01 DSM 32708; preferably in a CFU or weight ratio (DG® or LPC-S01: BbIBS01: BbIBS02: BlIBS01: LpIBS01: MIMBb23sg) of about 1:1:1:1:1:1, or (DG®: LPC-S01: BbIBS01: BbIBS02: BlIBS01: LpIBS01: MIMBb23sg) of about 1:1:1:1:1:1:1.

According to a further embodiment, mixture M of the composition of the invention comprises or, alternatively, consists of the inactivated strain of Bifidobacterium bifidum MIMBb23sg (or BbfIBS01) DSM 32708, preferably inactivated by gamma rays.

For example, the mixture M of the composition of the invention comprises or alternatively consists of strain of Bifidobacterium bifidum MIMBb23sg (or BbfIBS01) DSM 32708 and L. paracasei DG® CNCM I-1572 and/or L. paracasei LPC-S01® DSM 26760 (or (h) L. paracasei m.biome LIVESKIN88 DSM 33788 instead of (b) L. paracasei LPC-S01® DMS 26760), as inactivated strains (preferably by gamma ray), preferably in a CFU or weight ratio (MIMBb23sg: DG® or LPC-S01®) of about 1:1, or (MIMBb23sg: DG®:LPC-S01®) of about 1:1:1.

The composition of the invention, comprising said mixture M according to any embodiment of the present description (preferably comprising the inactivated strain, for example by gamma rays, L. paracasei DG® CNCM I-1572 and/or L. paracasei LPC-S01® DSM 26760 and/or Bifidobacterium bifidum MIMBb23sg (or BbfIBS01) DSM 32708), can further comprise at least one prebiotic, for example selected from: inulin, fructo-oligosaccharides (FOS), galacto-oligosaccharides (GOS), guar gum and mixtures thereof

Furthermore, the composition of the invention, comprising said mixture M according to any embodiment of the present description (preferably comprising the inactivated strains, for example by gamma rays, L. paracasei DG® CNCM I-1572 and/or L. paracasei LPC-S01® DSM 26760 and/or Bifidobacterium bifidum MIMBb23sg (or BbfIBS01) DSM 32708), can further comprise:
vitamins, such as those of B group, vitamin C, vitamin D, vitamin A and vitamin E; and/or
antioxidant substances such as glutathione, polyphenols such as resveratrol and trans-resveratrol, coenzyme Q10, astaxanthin, lycopene; and/or
plant extracts (botanicals), such as Echinacea, Uncaria tomentosa, fermented papaya, berries and ginger (Zingiber officinale); and/or
minerals, such as zinc, selenium, magnesium, iron, potassium, copper; and/or
amino acids, such as glutamine, arginine, tryptophan; and/or
omega-3 fatty acids.

The compositions or mixtures M of the present invention, according to any of the disclosed embodiments, can be prepared in the form of a pharmaceutical composition (or Live Biotherapeutic Products), a medical device composition (EU Reg. 2017/745), a food supplement, a food (or novel food or food for special medical purposes (AFMS)), a composition for a food supplement or food, or, alternatively, a composition for cosmetic use.

The compositions or mixtures M or strains of bacteria of the present invention, according to any of the disclosed embodiments, may be formulated for oral use (gastrointestinal or sublingual or buccal), for nasal inhalation (e.g., spray or drops), for oral inhalation. for topical dermal or vaginal or rectal or ocular use.

The compositions or mixtures M or strains of bacteria of the present invention formulated for oral use, may be in a solid form selected from: tablets, chewable tablets, orosoluble tablets, granules, flakes, soluble powder, orosoluble powder, capsules; or, alternatively, in a liquid form selected from: solutions, suspensions, dispersions, emulsions, dispensable liquid in the form of sprays, syrups, enemas; or, alternatively, in a semi-liquid form selected from: soft-gel, gel; preferably the composition of the invention is for oral use in a solid or liquid form.

Advantageously, each of said inactivated strains of bacteria of the present invention, preferably inactivated by gamma rays, are present in the composition or mixture M of the invention at a concentration expressed as Colony Forming Unit (CFU) in the range from about 0 CFU to 1×10⁷ CFU (e.g., 1×10 CFU, 1×10² CFU, 1×10³ CFU, 1×10⁴ CFU, 1×10⁵ CFU, or 1×10⁶ CFU) preferably from 10×10³ CFU to 10×10⁴ CFU.

These amounts in CFU can be referred to a dosage unit, administerable once or twice or three or four times a day.

Alternatively, these amounts in CFU can be referred to daily doses, which may be administered to the subject in need in as an individual single dose (single dose) or divided into repeated doses, e.g. two, three or four doses per day.

The composition of the invention, comprising said mixture M according to any of the embodiments of the present invention, can further comprise said at least one additive and/or excipient of pharmaceutical or food grade, i.e. a substance without therapeutic activity suitable for pharmaceutical or food use. In the context of the present invention, additives and/or excipients acceptable for pharmaceutical or food use include all auxiliary substances known to the person skilled in the art to be used in the preparation of compositions in solid, semi-solid or liquid form, such as, for example, diluents, solvents, solubilizers, acidifiers, thickeners, sweeteners, flavourings, dyes, sweeteners, lubricants, surfactants, preservatives, stabilisers, pH stabilising buffers and mixtures thereof; for example maltodextrin.

It is an object of the present invention the inactivated strains of bacteria of the present invention, preferably by gamma irradiation, and the compositions of the present invention (according to any of the disclosed embodiments) for use as a medicament in a subject in need, such as a mammalian, human or animal subject, preferably human.

Said inactivated strains of bacteria of the present invention, preferably by gamma irradiation, and compositions of the present invention (according to any of the described embodiments) are advantageously for use in a method of treating, preventively and/or curatively, a gastrointestinal disease and/or symptom of an inflammatory nature, preferably selected from: chronic inflammatory bowel disease (IBD), Crohn's disease, ulcerative colitis, microscopic colitis, celiac disease, diverticular disease, diverticulitis, Helicobacter pylori, peptic or gastric ulcer, and duodenal ulcer; preferably selected from: chronic inflammatory bowel disease (IBD), Crohn's disease, ulcerative colitis, microscopic colitis, diverticular disease, and diverticulitis.

Said inactivated bacterial strains of the present invention, preferably by gamma irradiation, and compositions of the present invention (according to any of the disclosed embodiments) are advantageously for use in a method of treating, preventatively and/or curatively, a gastrointestinal pathology and/or symptom of a functional nature, preferably selected from: irritable bowel syndrome (IBS), constipated IBS, diarrhoeal IBS, alternating IBS, unclassified IBS, dyspepsia, pyrosis, disorders affecting the oesophagus, stomach and duodenum, bacterial overgrowth syndrome (SIBO), gastrointestinal disorders with sub-inflammatory states.

Said inactivated strains of bacteria of the present invention, preferably by gamma irradiation, and the compositions of the present invention (according to any of the disclosed embodiments) are advantageously for use in a method of treating, preventively and/or curatively, a disease and/or symptom related to a de-regulation of the immune system, preferably autoimmune diseases, allergies, immunodeficiency diseases, skin diseases, such as acne, atopic dermatitis, at least one provoked or induced skin damage, acute or chronic skin inflammation or infection, bacterial or viral or fungal skin infection, abscess, apostema, empyema, phlegmon, paterecium, furuncle, carbuncle, hydrosadenitis, erysipelas, psoriasis, atopic dermatitis, acne, acute or chronic dermatosis, rosacea, couperose, erythema, reddening of the skin, burns, sunburn, reactivation of oral herpes, pressure lesions, ulcers, rhagades, fistula, sore, wound, contusion, abrasion, ecchymosis, haematoma, excoriation, keratosis, hyperkeratosis, keloid.

Examples of said at least one provoked or induced skin damage are: damage from UV radiation, damage from weather conditions adverse to the skin, preferably sunlight, cold or wind, and/or damage from living conditions harmful to the skin, preferably pollution, smoking or alcohol consumption.

Said inactivated bacterial strains of the present invention, preferably by gamma ray irradiation, and the compositions of the present invention (according to any of the disclosed embodiments) are advantageously for use in a method of treating, preventatively and/or curatively, inflammatory musculoskeletal disease, rheumatologic disease, inflammatory joint disease and inflammatory post-surgical disease, preferably osteoarthritis, rheumatoid arthritis and ankylosing spondylitis.

Said inactivated bacterial strains of the present invention, preferably by gamma irradiation, and the compositions of the present invention (according to any of the disclosed embodiments) are advantageously for use in a method of treating, preventively and/or curatively, a disease and/or symptom related to a de-regulation of serotonin production and/or uptake, preferably psychiatric disorders, such as anxiety, depression, obsessive-compulsive disorder (OCD), eating disorders and psychosis.

Said inactivated bacterial strains of the present invention, preferably by gamma irradiation, and compositions of the present invention (according to any of the disclosed embodiments) are advantageously for use in a method of treating, preventatively and/or curatively, neurological disorders, such as for example degenerative diseases (such as Alzheimer's or Parkinson's) or autism.

Said inactivated bacterial strains of the present invention, preferably by gamma irradiation, and compositions of the present invention (according to any of the disclosed embodiments) are advantageously for use in a method of treating, preventively and/or curatively, a pathology and/or symptom arising from a pathogenic microorganism, preferably wherein said pathogenic microorganism (e.g., bacterium or virus) is selected from: a strain of the group A Streptococcus (GAS), preferably a strain belonging to the species Streptococcus pyogenes, Coxsackievirus B5, Herpes Simplex Virus-1, Mumps Virus, Adenovirus-5, Influenza Virus, preferably Influenza Virus A/H1N1, Human Herpesvirus-6, Porcine Parvovirus, Porcine Reproductive and Respiratory Syndrome Virus, RNA virus, RNA virus SARS-CoV2 (COVID-19), DNA virus, Gram-positive bacteria, Gram-negative bacteria, Pseudomonas, Staphylococcus, Cutibacterium acnes.

Said inactivated bacterial strains of the present invention, preferably by gamma irradiation, and compositions of the present invention (according to any of the disclosed embodiments) are advantageously for use in a method of treating, preventatively and/or curatively, a disease and/or symptom associated with a significant increase in both primary and secondary pro-inflammatory cytokines (chemokines) (e.g. IL-6, IL-15, IL-12, IL-1α, IL-1β, IL-2, and TNF-α) and/or a significant decrease in anti-inflammatory cytokines (e.g. IL-10 and TGF-β), preferably diseases of: the locomotor system (muscular and skeletal system), the digestive system, the urinary system, the genital system, the respiratory system, the integumentary system, the immune system and the circulatory system.

In the context of the present invention, the term “significant” decreasing or increasing or modulation can, for example, means “significant from a statistical point of view” or “clinically significant”. Furthermore, “significant from a statistical point of view” (or simply “statistically significant”) can means p<0.1 or p<0.05 or p<0.01.

Said inactivated strains of bacteria of the present invention, preferably by gamma irradiation, and the compositions of the present invention (according to any of the disclosed embodiments, preferably the combination of the strains L. paracasei DG® CNCM I-1572 and LPC-S01® DMS 27260, such as the commercial product AminoAlta™) are advantageously for use in a method of treatment (therapeutic or non-therapeutic), preventive and/or curative, of diseases, symptoms or conditions related to a proteins and/or amino acids deficiency, for example in paediatric subjects, elderly, pregnant or lactating women, athletes and/or students. Examples of such diseases, symptoms or conditions related to a protein and/or amino acid deficiency are: reduced metabolic efficiency, reduced blood corpuscles, impaired healing processes, reduced muscle strength, reduced muscle mass, muscle depletion, weight loss, reduced athletic performance, premature fatigue, difficulty in concentrating, anxiety, sleep disorders, mood swings, increased susceptibility to infections, digestive disorders, glycaemic alterations, increased cholesterol, altered haematochemical parameters, malnutrition syndrome (such as Kwashiorkor or Biafra), osteoporosis, reduction of at least one protein-based component of an organism selected from nails, hair, skin, enzymes, neurotransmitters, hormones and immunoglobulins.

It is an object of the present invention a method of therapeutic, preventive and/or curative treatment of at least one disease or symptom described in the present description, wherein said method of treatment involves administering a therapeutically effective amount of at least one inactivated strain of bacteria of the present invention or a mixture or composition thereof to a subject in need thereof.

It is an object of the present invention the non-therapeutic use on healthy subjects (or cosmetic use) of the inactivated strains of bacteria of the present invention and mixtures or compositions thereof (according to any one of the embodiments), preferably inactivated by gamma rays, wherein said use is intended to maintain the balance of or strengthen the gut microbiota and/or immune system of healthy subjects to enable said subjects to perform physical or mental performances without impairing their physical or mental health, or to perform better physical or mental performances, or to quickly recover their standard physical or mental state after a physical or mental performance.

It is an object of the present invention the cosmetic use on healthy subjects of the inactivated strains of bacteria of the present invention and mixtures or compositions thereof (according to any one of the embodiments), preferably inactivated by gamma rays.

The inactivated strains of bacteria, preferably by gamma rays, of the present invention and compositions thereof can be administered to all categories of subjects in need, including the elderly, pregnant or lactating women, paediatric subjects (0-3 and/or 3-12 years), subjects suffering from comorbidities, (healthy) athletes and (healthy) students.

Based on the knowledge of the functional characteristics and use in therapeutic and non-therapeutic methods of treatment of non-inactivated strains (probiotics) L. paracasei DG® CNCM I-1572 and L. paracasei LPC-S01® DSM 26760, and based on the demonstration in the present invention that gamma-inactivated strains of bacteria are viable and have a cellular composition completely identical to non-inactivated (but non-culturable) strains, it follows that gamma-inactivated strains of L. paracasei DG CNCM I-1572 and L. paracasei LPC-S01 DSM 26760 can be useful and used in therapeutic and non-therapeutic methods of treatment where the strains L. paracasei DG® CNCM I-1572 and L. paracasei LPC-S01® DSM 26760 have been non-inactivated (probiotics).

L. paracasei DG® CNCM I-1572 and L. paracasei LPC-S01® DSM 26760 non-inactivated strains (probiotics) have been shown to have the ideal characteristics to strengthen the gut microbiota and modulate the appropriate inflammatory/immune pathways to be effective in a method of treating infection and/or inflammation of the gastrointestinal tract, autoimmune diseases, skin diseases, respiratory tract diseases, and/or genito-urinary tract diseases.

Furthermore, it has been observed that:
non-inactivated (probiotics) L. paracasei DG® CNCM I-1572 and/or L. paracasei LPC-S01® DSM 26760 survive during gastrointestinal transit and arrive alive and viable in the intestine. L. paracasei DG® CNCM I-1572 has been tested in recovery studies; recovery studies are the only demonstration that the strain is truly capable of crossing the gastrointestinal tract alive and colonising the intestine in humans;
not inactivated (probiotic) L. paracasei DG® CNCM I-1572 is able to positively modulate the structure/function of the intestinal microbiota, thus helping the balance of the intestinal microbiota by statistically significantly increasing Lactobacillus (which are reduced in patients with coronavirus infection) and reducing pathogenic and/or potentially pathogenic bacterial populations;
non-inactivated (probiotics) L. paracasei DG® CNCM I-1572 and/or L. paracasei LPC-S01® DSM 26760 are able to positively modulate inflammatory/immune pathways, inducing a statistically significant reduction in IL-1α, IL-15, IL-6 and IL-8 and a statistically significant increase in IL-10. For L. paracasei DG® CNCM I-1572, this effect could be attributable to the presence of its peculiar EPS (exopolysaccharide), which coats the bacterium like a natural ‘microencapsulation’;
not inactivated (probiotic) L. paracasei DG® CNCM I-1572 induces a statistically significant increase in faecal levels of short-chain fatty acids, such as acetate and especially butyrate.

It should be noted that the bacterial strain L. paracasei DG® (CNCM I-1572) was redeposited on 2 Feb. 2022 as Lacticaseibacillus paracasei DG I1572 DSM 34154 after the reclassification of the genus Lactobacillus published by Zheng et al. in the scientific journal Int. J. Syst. Evol. Microbiol., 70(4):2782-2858, 2020. The two designations mentioned above are interchangeable with each other because they always refer to the same strain of bacteria,

The strains of bacteria or compositions or mixtures M of the present invention, according to any of the disclosed embodiments, can be for use in methods of treatment as adjuvants to further therapeutic approaches.

It is an object of the present invention (or description) a process for preparing said inactivated strains of bacteria of the present invention (VBNC) by gamma irradiation, wherein said process comprises or alternatively consists of the steps performed in accordance with ISO111137 (sterilization of health care products-Radiation; ISO111137-1:2006, ISO111137-2:2012, ISO111137-3:2013)

It is an object of the present invention (or description) said inactivated strains of bacteria of the present invention (VBNC) obtainable by gamma irradiation, according to a process comprising or alternatively consisting of steps carried out in accordance with ISO111137.

Unless otherwise specified, the expression composition or mixture or other which includes a component in an amount “within a range from x to y” means that said component may be present in the composition or mixture or other in all amounts within said range, even if not explicitly stated, including the extremes of the range.

Unless otherwise specified, a statement that a composition or mixture “comprises” one or more components or substances means that other components or substances may be present in addition to the one or more specifically mentioned.

A “method of treatment” within the scope of the present invention means an intervention on a subject in need, comprising the administration of a therapeutically effective amount (according to the judgment of the person skilled in the art) of a composition or mixture of substances, having as its purpose the elimination, reduction/decrease or prevention of a pathology or disease and symptoms or disorders thereof.

The term “subject(s)” within the scope of the present invention refers to mammalian, human or animal subjects (e.g. pet animals such as dogs, cats, horses, sheep or cattle). Preferably, the compositions of the invention are for use in therapeutic or non-therapeutic methods of treatment on human subjects.

In the context of the present invention, it is not excluded that the properties of “gammated” cells, obtained by the methods described herein, may also be very different (not lower or higher, but just different) from non-gammated cells, but at the same time being of interest and in any case very useful.

FRn embodiment of the present invention are set out below.
FR1 An inactivated strain of bacteria,
in which said bacteria are preferably viable but not culturable,
in which said strain of bacteria belongs to the genus Lactobacillus or Bifidobacterium and is selected from the group comprising or, alternatively, consisting of: Lactobacillus paracasei, Lacticaseibacillus paracasei, Bifdobacterium bifidum, Bifidobacterium breve, Bifidobacterium animalis subsp. lactis, Lactobacillus plantarum and Lactiplantibacillus plantarum,
in which this strain of bacteria is inactivated by gamma irradiation.
FR2 The inactivated strain of bacteria according to FR1, wherein said inactivated strain of bacteria is selected from the group comprising or, alternatively, consisting of:
   Lacticaseibacillus paracasei DG® deposited at the National Collection of Cultures of Microorganisms of the Pasteur Institute in Paris with the accession number CNCM I-1572;
   Lacticaseibacillus paracasei LPC-S01® deposited at the Deutsche Sammlung von Mikroorganismen und Zelkulturen GmbH (DSMZ) with the accession number DSM 26760;
   Bifidobacterium bifidum MIMBb23sg (or BbfIBS01) deposited at the Deutsche Sammlung von Mikroorganismen und Zellkulturen GmbH (DSMZ) with deposit number DSM 32708;
   Bifidobacterium breve BbIBS01 deposited at the Deutsche Sammlung von Mikroorganismen und Zellkulturen GmbH (DSMZ) with deposit number DSM 33231;
   Bifidobacterium breve BbIBS02 deposited at the Deutsche Sammlung von Mikroorganismen und Zellkulturen GmbH (DSMZ) with deposit number DSM 33232;
   Bifidobacterium animalis subsp. lactis BlIBS01 deposited at the Deutsche Sammlung von Mikroorganismen und Zellkulturen GmbH (DSMZ) with deposit number DSM 33233;
   Lactiplantibacillus plantarum LpIBS01 deposited at the Deutsche Sammlung von Mikroorganismen und Zellkulturen GmbH (DSMZ) with deposit number DSM 33234; and
   Lacticaseibacillus paracasei m.biome LIVESKIN88 deposited at the Deutsche Sammlung von Mikroorganismen und Zellkulturen GmbH (DSMZ) with deposit number DSM 33788.
FR3 A composition comprising:
(i) a mixture M comprising or, alternatively, consisting of at least one strain of inactivated bacteria according to any one of the preceding claims, and optionally,
(ii) at least one additive and/or excipient of pharmaceutically acceptable grade.
FR4 The composition according to FR3, wherein said mixture M comprises or, alternatively, consists of an inactivated bacterial strain of Lacticaseibacillus paracasei DG® CNCM I-1572 and/or an inactivated bacterial strain of Lacticaseibacillus paracasei LPC-S01® DSM 26760 and/or an inactivated bacterial strain of Lacticaseibacillus paracasei m.biome LIVESKIN88 DSM 33788.
FR5 The composition according to FR3 or FR4, wherein said mixture M comprises or, alternatively, consists of an inactivated bacterial strain of Bifidobacterium bifidum MIMb23sg (or BbfIBS01) DSM 32708.
FR5 The composition according to FR4 or FR5, wherein said mixture M further comprises at least one strain of inactivated bacteria selected from the group consisting of: Bifidobacterium breve BbIBS01 DSM 33231, Bifidobacterium breve BbIBS02 DSM 33232, Bifidobacterium animalis subsp. lactis BlIBS01 DSM 33233, Lactiplantibacillus plantarum LpIBS01 DSM 33234, Bifidobacterium bifidum MIMBb23sg=BbfIBS01 DSM 32708, and mixtures thereof.
FR7 The inactivated strain of bacteria or composition according to any of the preceding FRs, wherein said strain of bacteria or said composition is formulated for oral use, nasal inhalation, oral inhalation, topical dermal use, topical vaginal use, topical rectal use or topical ocular use.
FR8 The inactivated strain of bacteria or composition according to any of the preceding FRs for use as a medicament in a subject in need, wherein said subject is a mammal, preferably a human subject.
FR9 The inactivated strain of bacteria or composition according to any of the preceding FRs for use in a method of treating a gastrointestinal disease or symptom of an inflammatory or functional nature in a subject in need;
   wherein preferably said gastrointestinal disease or symptom of an inflammatory nature is selected from chronic inflammatory bowel disease (IBD), Crohn's disease, ulcerative colitis, microscopic colitis, diverticular disease, diverticulitis, Helicobacter pylori, peptic or gastric ulcer, and duodenal ulcer; preferably selected from: chronic inflammatory bowel disease (IBD), Crohn's disease, ulcerative colitis, microscopic colitis, diverticular disease, and diverticulitis;
   wherein preferably said gastrointestinal disease or symptom of a functional nature is selected from: irritable bowel syndrome (IBS), constipated IBS, diarrhoeal IBS, alternating IBS, unclassified IBS, dyspepsia, pyrosis, disorders of the oesophagus, stomach and duodenum, bacterial overgrowth syndrome (SIBO), gastrointestinal disorders with sub-inflammatory states.

### Examples

An example of a composition according to the invention is a product comprising a mixture of gamma rays inactivated strains, such as (a) L. paracasei DG® CNCM I-1572 and (b) L. paracasei LPC-S01® DSM 26760, i.e. a product similar in composition to the commercial product AminoAlta™ (Sofar Americas, Inc.). The AminoAlta™ product (a product not according to the invention) comprises a mixture of non-inactivated strains, such as (a) L. paracasei DG® CNCM I-1572 (freeze-dried powder), (b) L. LPC-S01® DSM 26760 (freeze-dried powder) and maltodextrin, according to Table 1 and Table 2.

TABLE 1

| | | |
|---|---|---|
| Ingredients list | Qty per 100 g | Qty per unit |

| | | | | |
|---|---|---|---|---|
| Maltodextrin | 92.500 | g | 1850.000 | mg |
| Lacticaseibacillus paracasei | 3.750 | g | 75.000 | mg |
| LPC-S01 ® DSM 26760 | | | | |
| Lacticaseibacillus paracasei | 3.750 | g | 75.000 | mg |
| DG ® CNCM I-1572 | | | | |
| Total | 100.000 | g | 2000.000 | mg |

TABLE 2
NUTRIENT SUPPLY

| Average content | per 100 g | | Per daily dose |
|---|---|---|---|
| Total ferment count | 1500.000 | bn. U.F.C .* | 30.000 billion. U.F.C .* |
| of which Lacticaseibacillus paracasei | 750.000 | bn. U.F.C .* | 15.000 billion. U.F.C .* |
| LPC-S01 ® DSM 26760 | | | |
| of which Lacticaseibacillus paracasei | 750.000 | bn. U.F.C .* | 15.000 billion. U.F.C .* |
| DG ® CNCM I-1572 | | | |

*Amount of ferments in the formula

### Experimental Part

1. Purpose

Evaluating the characteristics (e.g., adhesive capacity to the intestinal membrane, β-galactosidase activity, etc.) and cellular composition of strains of bacteria and composition thereof according to the present invention, wherein said strains of bacteria are inactivated by gamma rays, for example at different intensities of ionising radiation.

Said characteristics and the cellular composition of gamma rays inactivated bacterial strains were compared with those of similar non-inactivated bacterial strains and heat-inactivated bacterial strains.

2. Materials

Samples analysed:
Combination comprising Lacticaseibacillus paracasei DG® CNCM I-1572 and Lacticaseibacillus paracasei LPC-S01 DSM 26760® (according to Tables 1 and 2), abbreviated “Combination DG®+LPC-S01”, treated according to Table 3.
Bifidobacterium bifidum MIMBb23sg (or BbfIBS01) DSM 32708, treated according to Table 3.

TABLE 3

| Sample | Inactivation treatment |
|---|---|
| Combination DG ® + LPC-S01 ‘live’ (AminoAlta ™) | no treatment |
| Combination DG ® + LPC-S01 “TT” | Heat treated (80° C. × 30 minutes) |
| Combination DG ® + LPC-S01 “γ” | Gamma rays irradiated (in accordance with |
| | ISO 11137; from 6 kGy to 25 kGy) |
| Bifidobacterium bifidum MIMBb23SG ‘live’ | no treatment |
| Bifidobacterium bifidum MIMBb23SG ‘TT’ | Heat treated (80° C. × 30 minutes) |
| Bifidobacterium bifidum MIMBb23SG ‘γ’ | Gamma rays irradiated (in accordance with |
| | ISO 11137; from 6 kGy to 25 kGy) |

3. Methods

3.1. Assessment of Adhesion on Caco-2

In this assay, a known amount of bacterial cells is placed on a layer of Caco-2, a line consisting of human colorectal adenocarcinoma epithelial cells that under appropriate growth conditions develop morphofunctional characteristics typical of enterocytes with an absorptive role in the small intestine. This is a cell culture line commonly used as a model to assess the ability of probiotics to adhere to human intestinal epithelial cells.

Specifically, Caco-2 cells were grown on coverslips placed in the wells of multiwell plates with Dulbecco's Modified Eagle's Medium (DMEM) supplemented with 10% (v/v) heat-inactivated fetal bovine serum, 1% sodium pyruvate, 100 U/ml penicillin, 100 mg/ml streptomycin, 0.1 mM non-essential amino acids, 2 mM L-glutamine (all reagents from Merck Life Science) and incubated at 37° C. in a 5% carbon dioxide atmosphere.

Caco-2 cells were used for adhesion tests 15 days after confluence, i.e. when fully differentiated, as they express in such state tight junctions, microvilli and a number of enzymes and transporters specific to small intestinal enterocytes. During this period of differentiation, the culture medium was changed every two days with fresh complete medium. On the day of the adhesion test, the cell monolayers were washed thoroughly twice with phosphate buffered saline (PBS) pH 7.3 pre-warmed at 37° C., in order to remove non-adherent cells before the addition of the bacterial cells. Following counting, approximately 2×10⁸ bacterial cells resuspended in PBS were taken for each bacterial strain and incubated with the Caco-2 cell monolayer for 1 hour at 37° C. and 5% carbon dioxide.

The monolayers were washed three times with PBS in order to specifically release non-adherent bacteria and incubated with 2 ml methanol for 8 min at room temperature to fix the cells. Subsequently, cells were stained with 3 ml Giemsa solution (1:20; Carlo Erba, Milan, Italy) and left 30 min in the dark at room temperature.

Finally, the wells were washed with PBS until the dye in excess was completely removed, the coverslips were removed from the plates and placed on coverslips in Petri dishes, then dried in an incubator at 30° C. for 30 minutes. The slides were then examined microscopically at 40× or 100× magnification in immersion oil.

Using this method, it is possible to quantify the adhesion capacity of probiotic strains by counting using a microscope the number of bacterial cells attached to the layer, counting then the number of bacterial cells and Caco-2 nuclei present in each field of view of the microscope (10 fields of view for each condition) and the number of attached bacterial cells was reported on 100 Caco-2 cell nuclei, obtaining a quantitative parameter known as the adhesion index.

3.2. Evaluation of β-Galactosidase Activity (Metabolic Activity)

The β-galactosidase activity of the test products was assessed by colorimetric assay on protein extract obtained from cell lysis of the lyophilised final product. In detail, 1 g of lyophilised product was resuspended, washed twice in 20 ml of 0.1 M phosphate buffer pH 7 and resuspended in a final volume of 5 mL. Following cell disruption achieved by beat beater (4 cycles×20 seconds×6300 RPM), the suspension was centrifuged at 13,000 rpm for 45 minutes at 5° C.

After quantification by Bradford assay, a 100 μL aliquot of the protein extract was added to 130 μL of phosphate buffer and 100 μL of o-nitrophenyl-β-galactosidase (ONPG) (3 mg/mL), and incubated for 2 hours at 37° C. while monitoring the change in absorbance at 420 nm.

Enzyme activity was reported as a relative unit, derived by comparing the enzyme units of the analysed sample with the enzyme units of the “non-inactivated” sample of the respective products.

3.3. Cytofluorimetric Analysis of the Composition of the Preparation

The viability of the bacteria present in the product under analysis was assessed by cytofluorimetry using the BD Accuri™ C6 flow cytometer (BD Biosciences, Milan, Italy).

Cytofluorimetric analysis provides information on the number of living, damaged and dead cells of the different products under analysis.

The term ‘live cells’ is used to refer to cells with a substantially intact membrane that show active fluorescence in green following marking with SYT024+propidium iodide.

The term ‘damaged cells’ is used to describe cells with a slightly damaged membrane that show fluorescence in both green and red after marking with SYT024+propidium iodide.

The term ‘dead cells’ refers to cells with totally damaged membranes that show red fluorescence following marking with SYT024+propidium iodide.

The composition in terms of live, damaged and dead cells of the lyophilised test product was made from the suspension of 1 g of lyophilisate resuspended in PBS buffer to give a 1:10 dilution. Following homogenisation, the cell suspension was subjected to serial decimal dilutions in filtered PBS buffer in 1.5 ml eppendorf tubes. These dilutions were used for viability determination by setting the following instrumental parameters:
Sample acquisition volume: 50 μl
Threshold: FSC: 4000; SSC: 1000

The cytofluorimetric analysis included an initial acquisition of the unmarked sample in order to identify the decimal dilution suitable for subsequent marking, verifying that the number of events/μl detected was in the range of 1000 to 3000.

After establishing the correct decimal dilution, the cells were marked by adding 50 μl of the sample and 10 μl of SYTO™ 24 marker (absorption (nm): DNA(490), RNA(ND))+propidium iodide (PI) to 440 μl of diluent (filtered PBS).

The total number of viable cells was then identified by double-tagging the samples according to protocol B of the ISO 19344:2015 method (IDF 232), the principle of which is based on the use of these two fluorophores. Specifically, SYTO® 24 is a dye that permeates the membrane of all cells by binding to the nucleic acids, making them green fluorescent, while PI is a dye that permeates only bacteria whose membranes are damaged, making the cells red fluorescent. In the presence of both fluorophores, the red fluorescence of PI causes a reduction in the green fluorescence of SYTO™ 24. Thus, live bacteria with intact cell membranes have green fluorescence (active fluorescent cells), bacteria with damaged membranes show both green and red fluorescence (damaged fluorescent cells) and bacteria with ruptured membranes show red fluorescence (inactive fluorescent cells).

4. Results

4.1. Evaluation of the Adhesive Capacity of Samples Comprising Inactivated Strains of Bacteria.

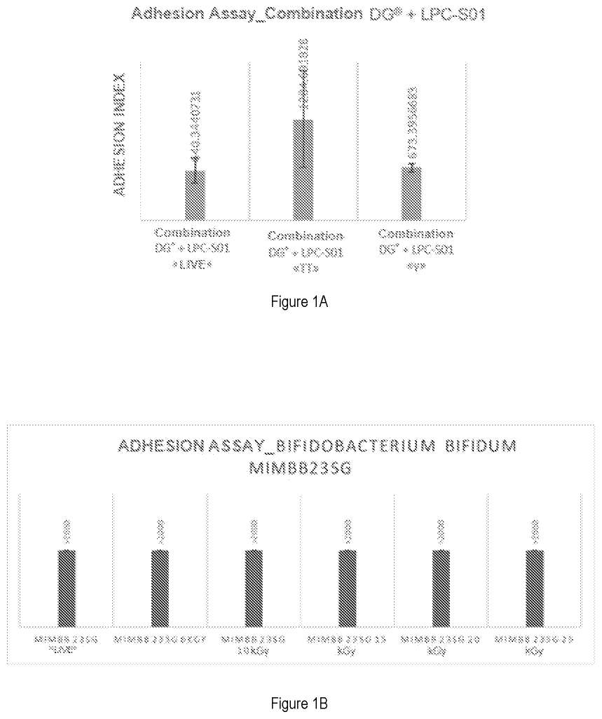

Samples comprising strains of bacteria inactivated by heat treatment (80° C. for 30 minutes) or by gamma irradiation (25 kGy per FIG. 1A) were evaluated and compared with a sample comprising the same strains of bacteria as non-inactivated (viable and culturable) strains of bacteria.

The results, shown in FIG. 1A, show how these inactivation treatments (i.e. heat treatment and gamma irradiation) have a different impact on the adhesive capacities of the samples analysed. The sample subjected to gamma irradiation inactivation (Combination DG®+LPC-S01 “γ”) preserves the adhesive properties of the non-inactivated blend AminoAlta Blend™ i.e. including viable and culturable bacterial strains.

The results, shown in FIG. 1B, show that the strain Bifidobacterium bifidum MIMBb23sg DSM 32708 subjected to gamma irradiation inactivation (6 kGy to 25 kGy) preserves the adhesive properties of the non-inactivated strain MIMBb23SG DSM 32708 (i.e. viable and culturable bacteria strain).

It can be seen that heat treatment causes a statistically significant decrease in the bacterium's adhesive capacity. On the contrary, irradiation treatment with “y” gamma rays does not cause a significant loss of the adhesive capacity of the probiotic strain under examination.

4.2. Evaluation of β-Galactosidase Activity of Test Samples

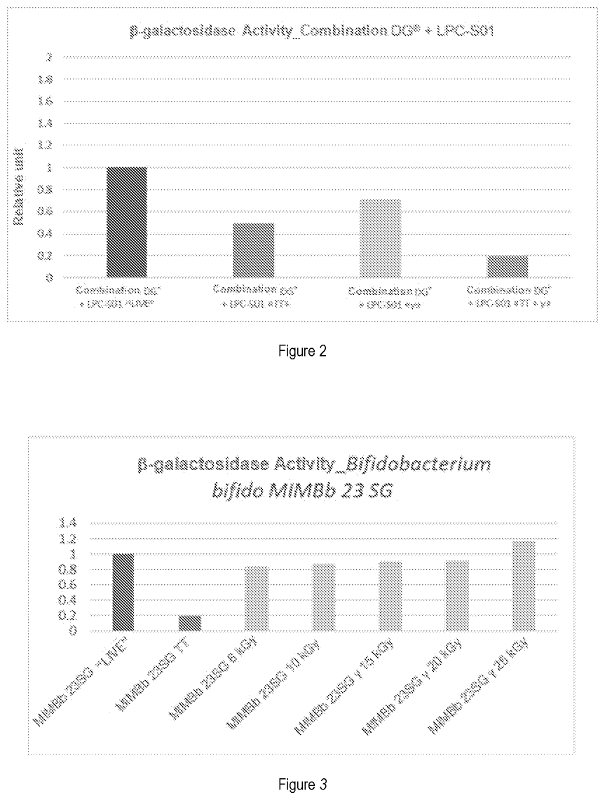

The results shown in FIGS. 2 and 3 show that exposure to elevated temperatures causes a marked decrease in β-galactosidase activity in both test samples.

To support this hypothesis, FIG. 2 shows the results obtained following the evaluation of the enzyme activity in question, of a preparation initially inactivated by ionising radiation (Composition DG®+LPC-S01 “γ”) and subjected to further heat treatment (Composition DG®+LPC-S01 “TT”+γ”), wherein the heat treatment is carried out at 80° C. for 30 minutes and the treatment with ionised radiation at 15 kGy.

The results show a loss of enzyme activity comparable to the loss obtained by subjecting the ‘live’ product to heat treatment (‘TT’).

Similar data were obtained from analyses carried out on Bifidobacterium bifidum MIMBb23sg DSM 32708 (FIG. 3), where it is particularly evident that the effect of heat treatment (80° C. for 30 minutes) is the cause of the decrease in β-galactosidase activity. On the contrary, in products irradiated with different intensities of gamma (“γ”) rays, the enzymatic activity is maintained unaltered with values comparable to those obtained from the analysis of the untreated product (MIMBb23SG “live”).

4.3. Cytofluorimetric Analysis of Test Samples

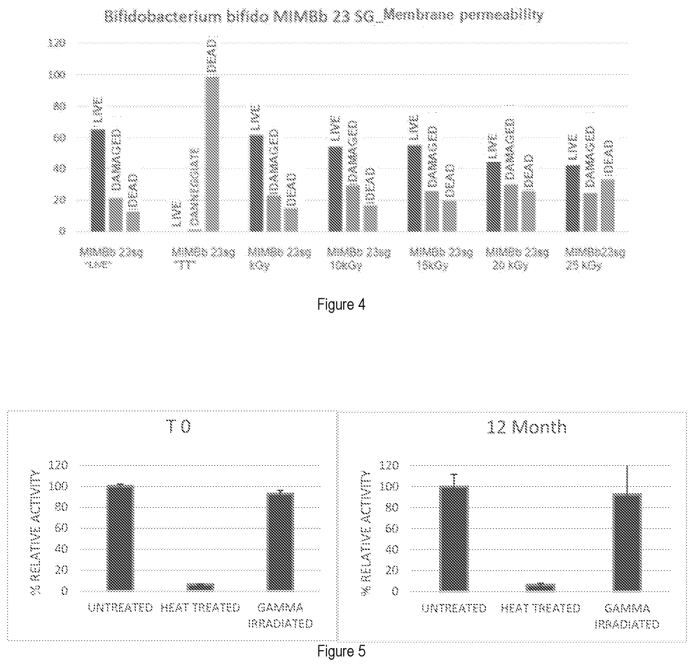

The samples analysed in section 4.2. above were used to perform cytofluorimetric analysis to determine the cellular composition of the sample in terms of living, damaged and dead cells (FIG. 4).

The results show that the sample subjected to treatment with ionising radiation (gamma rays) is able to preserve the characteristics of the cell membrane that regulate the level of permeability. In fact, despite the irradiated sample (gamma rays) is not culturable, as it is not able to form a colony on solid medium, the data obtained from cytofluorimetric analysis show that it is viable, highlighting a membrane integrity that is not significantly different from the untreated sample (non-inactivated strain).

On the contrary, data obtained from the analysis of samples comprising heat-treated bacterial strains show that this method is more destructive, causing both the loss of viability and the loss of characteristics that regulate membrane permeability.

The same method was used to assess the membrane status of probiotic strains belonging to both the Lactobacillus and Bifidobacterium genus in order to verify the effects of inactivation treatment on other strains of bacteria, different from those specifically described and studied herein. The result obtained is that the percentage of cells with intact cell membranes is very similar for all the strains of bacteria examined (not only those specifically described and studied herein) and present in the untreated sample and in the sample subjected to ionising radiation.

Evaluation of the Stability of Preparations Inactivated by Ionising Radiation

The stability of the products inactivated by ionising radiation was assessed by monitoring the development of β-galactosidase activity and membrane integrity over time.

Stability of β-Galactosidase Activity of Inactivated Products

The results shown in FIG. 5 show that the β-galactosidase activity of the Bifidobacterium bifidum MIMBb23sg preparation remains unaltered after 12 months.

As in previous tests, heat treatment causes a high decrease in enzyme activity. In contrast, inactivation by gamma irradiation does not appear to significantly affect β-galactosidase activity.

Evaluation of Membrane Integrity of Inactivated Products

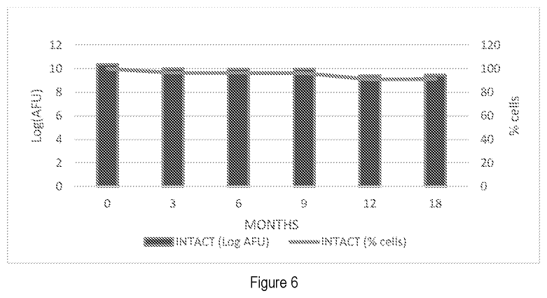

Experimental tests were set up to assess the stability over time of the integrity of the cell membrane preparation of Aminoalta™ inactivated by ionising radiation. In particular, the status of the cell membrane over time was assessed by cyto-fluorimetric analysis (FIG. 6).

The results show that the irradiated sample of Aminoalta™ is stable up to 18 months. In fact, from the analysis shown in FIG. 4, it can be seen that the number of cells with intact cell membranes is almost completely unchanged both in numerical terms (LOG AFU) and in percentage terms.

1. An inactivated strain of bacteria,
wherein these bacteria are preferably viable but not culturable,
wherein said strain of bacteria belongs to the genus Lactobacillus or Bifidobacterium and is selected from the group consisting of: Lactobacillus paracasei, Lacticaseibacillus paracasei, Bifidobacterium bifidum, Bifidobacterium breve, Bifidobacterium animalis subsp. lactis, Lactobacillus plantarum and Lactiplantibacillus plantarum,
wherein said strain of bacteria is inactivated by gamma irradiation, wherein said inactivated strain of bacteria is selected from the group consisting of:
Lacticaseibacillus paracasei DG® deposited at the National Collection of Cultures of Microorganisms of the Pasteur Institute in Paris with the accession number CNCM I-1572 (or Lacticaseibacillus paracasei DG I1572 DSM 34154);
Lacticaseibacillus paracasei LPC-S01® deposited at the Deutsche Sammlung von Mikroorganismen und Zellkulturen GmbH (DSMZ) with the accession number DSM 26760;
Bifidobacterium bifidum MIMBb23sg (or BbfIBS01) deposited at the Deutsche Sammlung von Mikroorganismen und Zellkulturen GmbH (DSMZ) with deposit number DSM 32708;
Bifidobacterium breve BbIBS01 deposited at the Deutsche Sammlung von Mikroorganismen und Zellkulturen GmbH (DSMZ) with deposit number DSM 33231;
Bifidobacterium breve BbIBS02 deposited at the Deutsche Sammlung von Mikroorganismen und Zellkulturen GmbH (DSMZ) with deposit number DSM 33232;
Bifidobacterium animalis subsp. lactis BlIBS01 deposited at the Deutsche Sammlung von Mikroorganismen und Zellkulturen GmbH (DSMZ) with deposit number DSM 33233;
Lactiplantibacillus plantarum LpIBS01 deposited at the Deutsche Sammlung von Mikroorganismen und Zellkulturen GmbH (DSMZ) with deposit number DSM 33234; and
Lacticaseibacillus paracasei m.biome LIVESKIN88 deposited at the Deutsche Sammlung von Mikroorganismen und Zellkulturen GmbH (DSMZ) with deposit number DSM 33788.

2. A composition comprising:
(i) a mixture M consisting of at least one inactivated strain of bacteria according to claim 1, and optionally,
(ii) at least one additive and/or excipient of pharmaceutically acceptable grade.

3. The composition according to claim 2, wherein said mixture M comprises an inactivated bacterial strain of Lacticaseibacillus paracasei DG® CNCM I-1572 (or Lacticaseibacillus paracasei DG I1572 DSM 34154) and/or an inactivated bacterial strain of Lacticaseibacillus paracasei LPC-S01® DSM 26760 and/or an inactivated bacterial strain of Lacticaseibacillus paracasei m.biome LIVESKIN88 DSM 33788.

4. The composition according to claim 2, wherein said mixture M comprises an inactivated bacterial strain of Bifidobacterium bifidum MIMBb23sg (or BbfIBS01) DSM 32708.

5. The composition according to claim 3, wherein said mixture M further comprises at least one other strain of inactivated bacteria selected from the group consisting of: Bifidobacterium breve BbIBS01 DSM 33231, Bifidobacterium breve BbIBS02 DSM 33232, Bifidobacterium animalis subsp. lactis BlIBS01 DSM 33233, Lactiplantibacillus plantarum LpIBS01 DSM 33234, Bifidobacterium bifidum MIMBb23sg=BbfIBS01 DSM 32708, and mixtures thereof.

6. The composition according to claim 2, wherein said mixture M consists of at least one inactivated bacterial strain of Lacticaseibacillus paracasei DG® CNCM I-1572 (or Lacticaseibacillus paracasei DG 11572 DSM 34154) and/or one inactivated bacterial strain of Lacticaseibacillus paracasei LPC-S01® DSM 26760 and/or one inactivated bacterial strain of Lacticaseibacillus paracasei m.biome LIVESKIN88 DSM 33788.

7. The composition according to claim 2, wherein said mixture M consists of at least one inactivated bacterial strain of Lacticaseibacillus paracasei DG® CNCM I-1572 (or Lacticaseibacillus paracasei DG 11572 DSM 34154) and/or an inactivated bacterial strain of Lacticaseibacillus paracasei LPC-S01® DSM 26760 and/or an inactivated bacterial strain of Lacticaseibacillus paracasei m.biome LIVESKIN88 DSM 33788 and/or Bifidobacterium bifidum MIMBb23sg (or BbfIBS01) DSM 32708.

8. The composition according to claim 6, wherein said mixture M further comprises at least one strain of inactivated bacteria selected from the group consisting of: Bifidobacterium breve BbIBS01 DSM 33231, Bifidobacterium breve BbIBS02 DSM 33232, Bifidobacterium animalis subsp. lactis BlIBS01 DSM 33233, Lactiplantibacillus plantarum LpIBS01 DSM 33234, Bifidobacterium bifidum MIMBb23sg=BbfIBS01 DSM 32708, and mixtures thereof.

9. The inactivated strain of bacteria or the composition according to claim 1, wherein said strain of bacteria or said composition is formulated for oral use, nasal inhalation, oral inhalation, topical dermal use, topical vaginal use, topical rectal use or topical ocular use.

10. The inactivated strain of bacteria or the composition according to claim 1 for use as a medicament in a subject in need thereof, wherein said subject is a mammal, preferably a human subject.

11. The inactivated strain of bacteria or the composition according to claim 1 for use in a method of treating a gastrointestinal disease or symptom of an inflammatory or functional nature in a subject in need thereof;
preferably wherein said gastrointestinal disease or symptom of an inflammatory nature is selected from chronic inflammatory bowel disease (IBD), Crohn's disease, ulcerative colitis, microscopic colitis, diverticular disease, diverticulitis, Helicobacter pylori, peptic or gastric ulcer, and duodenal ulcer; preferably selected from: chronic inflammatory bowel disease (IBD), Crohn's disease, ulcerative colitis, microscopic colitis, diverticular disease, and diverticulitis;
preferably wherein said gastrointestinal pathology or symptom of a functional nature is selected from: irritable bowel syndrome (IBS), constipated IBS, diarrhoeal IBS, alternating IBS, unclassified IBS, dyspepsia, pyrosis, disorders of the oesophagus, stomach and duodenum, bacterial overgrowth syndrome (SIBO), gastrointestinal disorders with sub-inflammatory states.

12. A method of treating a gastrointestinal disease or symptom of an inflammatory or functional nature in a subject in need thereof, comprising:
administering a composition of claim 1 to the subject.

13. The method of claim 12, wherein said subject has a gastrointestinal disease or symptom of an inflammatory nature selected from chronic inflammatory bowel disease (IBD), Crohn's disease, ulcerative colitis, microscopic colitis, diverticular disease, diverticulitis, Helicobacter pylori, peptic or gastric ulcer, and duodenal ulcer; preferably selected from: chronic inflammatory bowel disease (IBD), Crohn's disease, ulcerative colitis, microscopic colitis, diverticular disease, and diverticulitis

14. The method of claim 12, wherein said subject has a gastrointestinal disease or symptom of an inflammatory nature selected irritable bowel syndrome (IBS), constipated IBS, diarrhoeal IBS, alternating IBS, unclassified IBS, dyspepsia, pyrosis, disorders of the oesophagus, stomach and duodenum, bacterial overgrowth syndrome (SIBO), gastrointestinal disorders with sub-inflammatory states.

15. The method of claim 12, wherein said composition comprises an inactivated bacterial strain of Lacticaseibacillus paracasei DG® CNCM I-1572 (or Lacticaseibacillus paracasei DG I1572 DSM 34154) and/or an inactivated bacterial strain of Lacticaseibacillus paracasei LPC-S01® DSM 26760 and/or an inactivated bacterial strain of Lacticaseibacillus paracasei m.biome LIVESKIN88 DSM 33788.

16. The method of claim 12, wherein said composition comprises an inactivated bacterial strain of Bifidobacterium bifidum MIMBb23sg (or BbfIBS01) DSM 32708.

17. The method of claim 16, wherein said composition further comprises at least one other strain of inactivated bacteria selected from the group consisting of: Bifidobacterium breve BbIBS01 DSM 33231, Bifidobacterium breve BbIBS02 DSM 33232, Bifidobacterium animalis subsp. lactis BlIBS01 DSM 33233, Lactiplantibacillus plantarum LpIBS01 DSM 33234, Bifidobacterium bifidum MIMBb23sg=BbfIBS01 DSM 32708, and mixtures thereof.

18. The method of claim 12, wherein said composition consists of at least one inactivated bacterial strain of Lacticaseibacillus paracasei DG® CNCM I-1572 (or Lacticaseibacillus paracasei DG I1572 DSM 34154) and/or one inactivated bacterial strain of Lacticaseibacillus paracasei LPC-S01® DSM 26760 and/or one inactivated bacterial strain of Lacticaseibacillus paracasei m.biome LIVESKIN88 DSM 33788.

19. The method of claim 12, wherein said composition consists of at least one inactivated bacterial strain of Lacticaseibacillus paracasei DG® CNCM I-1572 (or Lacticaseibacillus paracasei DG I1572 DSM 34154) and/or an inactivated bacterial strain of Lacticaseibacillus paracasei LPC-S01® DSM 26760 and/or an inactivated bacterial strain of Lacticaseibacillus paracasei m.biome LIVESKIN88 DSM 33788 and/or Bifidobacterium bifidum MIMBb23sg (or BbfIBS01) DSM 32708.

20. The method of claim 12, wherein said strain of bacteria or said composition is formulated for oral use, nasal inhalation, oral inhalation, topical dermal use, topical vaginal use, topical rectal use or topical ocular use.
